(19) United States
(12) Patent Application Publication
(10) Pub. No.: US 2006/0198914 A1
(43) Pub. Date: Sep. 7, 2006

(54) Use of rutin and isoharmnetin for treating depressive states and depression and other emotion disorders

(75) Inventors: Shyam S. Chatterjee, Karlsruhe (DE); Michael Nöldner, Karlsruhe (DE); Karl Schötz, Durmersheim (DE)

Correspondence Address: EDWARDS & ANGELL, LLP, P.O. BOX 55874, BOSTON, MA, 02205, US

(73) Assignee: Bioplanta Arzneimittel GmbH, Ettlingen (DE)

(21) Appl. No.: 10/548,211

(22) PCT Filed: Mar. 2, 2004
(86) PCT No.: PCT/EP04/02085

§ 371 Date: Sep. 2, 2005

(30) Foreign Application Priority Data
Mar. 3, 2003 (DE) 103 09 235.8
Apr. 2, 2003 (DE) 103 15 022.6
Oct. 28, 2003 (DE) 103 50 194.0

(51) Int. Cl. A61K 36/33; A61K 31/7048; A61K 31/353

(52) U.S. Cl. 424/767; 514/27; 514/456

(57) ABSTRACT

The present invention relates to the use of rutin and isorhamnetin for the treatment of episodes of depression and depressive diseases as well as in case of other affective disorders and preliminary stages thereof.

With a one-year prevalence rate of about 10% and a lifetime prevalence rate of about 20% depressions are among the most common diseases in the Western industrial nations. They seriously affect the patient in his private and professional sector, irritate his surrounding, burden our health care system quite tremendously by way of an increased utilization of the primary medical care and many lost working days due to sick certificates and are even fatal in individual cases. Today's estimations assume that more than two third of the about 10,000 suicides a year in Germany are attributed to depressions. Depressions are clinically classified as follows (H. J. Möller, Der Internist 2000, 70):
1. Endogenic depression, unipolar and bipolar
2. Depressive neurosis
3. Reactive depression
4. Depression in case of schizoaffective psychosis
5. Organically based depression
6. Depressive symptoms in case of dementia.

Furthermore, depressive diseases are classified on the basis of their level of severity into weak, moderate or severe.

Preliminary stages of depressive diseases can manifest themselves in abjectness, feeling of reluctance, melancholia, unmotivation, labile or saddened mood or limitations of the emotional well-being.

Despite a plurality of different hypotheses the causes of depressive diseases are reconnoitered insufficiently. By way of example the following groups of medicaments are suitable for the medicinal treatment of depression and other affective disorders (H. J. Möller, Der Internist 2000, 70):
1. Selective serotonin reuptake inhibitors (SSRIs)
2. Selective noradrenaline reuptake inhibitors (SNRIs)
3. Selective serotonin and noradrenaline reuptake inhibitors (SSNRIs)
4. Tricyclic antidepressives (TCA)
5. MAO-A inhibitors
6. Other synthetic preparations
7. Phytopharmaceuticals

The common feature of all methods of treatment indicated above is that they do not become effective until 10 to 14 days of treatment, if at all. For about 20% of the patients the medicinal therapy is insufficient also in case of a combination of different preparations. Furthermore, all methods of treatment mentioned above exhibit the drawback of causing undesired side effects that often result in withdrawing the therapy. Therefore, there is a significant need for providing further agents for the treatment of depressive diseases and other affective disorders and the preliminary stages thereof, particularly agents which overcome one or more of the drawbacks indicated above totally or partly.

Thus, it is the object underlying the present invention to provide such agents.

According to the present invention, this object is solved by the use of rutin, isorhamnetin, isorhamnetin derivatives or plant parts and/or extracts produced therefrom, which contain rutin or isorhamnetin in a free or bonded form or from which isorhamnetin is formed by metabolization, (for the manufacture of a medicament or a dietetic food product) for the treatment of episodes of depression and depressive diseases or preliminary stages of other affective disorders as well as by a medicament and a dietetic food product for the treatment or for supporting the treatment of episodes of depression and depressive diseases or preliminary stages of other affective disorders, characterized by a content of rutin, isorhamnetin, isorhamnetin derivatives or plant parts and/or extracts produced therefrom which contain rutin or isorhamnetin in a free or bonded form or from which isorhamnetin is formed by metabolization as well as by a preparation as an oral administration form which further contains suitable pharmaceutically acceptable adjuvants.

Rutin (3-[[6-O-(6-deoxy-α-L-mannopyranosyl)-β-D-glucopyranosyl]oxy]-2-(3,4-dihydroxyphenyl)-5,7-dihydroxy-4H-1-benzopyran-4-one) and isorhamnetin (3,5,7-trihydroxy-2-(4-hydroxy-3-methoxyphenyl)-4H-1-benzopyran-4-one) pertain to the group of flavones and are found as ingredients in various plants. Moreover, isorhamnetin can be formed in the animal and human organism from other flavones by way of metabolic conversion. As a result of this metabolism, detectable isorhamnetin plasma levels can also be achieved by the uptake of plants which themselves do not contain isorhamnetin. An antidepressive effect has hitherto been shown neither for rutin nor for isorhamnetin.

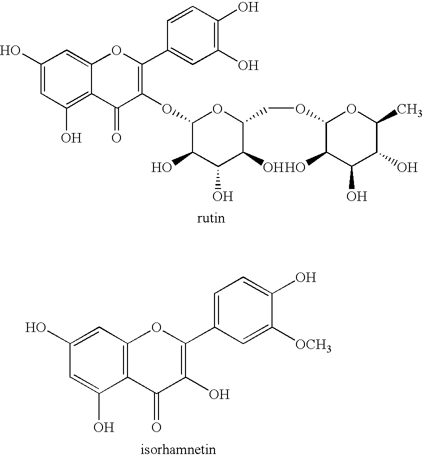

It has surprisingly been found that rutin, isorhamnetin, isorhamnetin derivatives or plant parts and/or extracts produced therefrom which contain at least one of the substances indicated above in a sufficient amount or which release isorhamnetin metabolically or hydrolytically, exhibit a significant antidepressive effect in an animal model. Isorhamnetin derivatives can be, for example, glycosides of isorhamnetin. Those extracts which contain at least one of the substances indicated above in a sufficient amount exhibit a content of 0.2% to 100%, preferably 1% to 10% rutin, isorhamnetin and/or isorhamnetin derivatives, wherein the transition of highly enriched special extracts to the pure substance (approximately 100%) does not exhibit a gap.

Such an effect has hitherto not been described for rutin, isorhamnetin and the plants and extracts indicated below and could not be expected in view of the hitherto known pharmacological and clinical effects.

Examples for plants that can be used according to the present invention are Allium cepa, Anethum graveolens, Brassica oleracea, Cassia senna, Crateagus species, Eucalyptus species, Filipendula ulmaria, Fagopyrum esculentum, Fagopyrum tataricum, Opuntia species, preverably Opuntia ficus-indica, Peumus boldus, Primula veris, Ruta graveolens, Sambucus nigra, Selenicereus grandilflorus and Sophora japonica. However, the present invention is not limited to these plants.

The extracts can be obtained according to per se known preparation methods in variable compositions, using solvents such as water, methanol, ethanol, acetone and the like as well as mixtures thereof at temperatures from room temperature to 100° C. under slight to vigorous mixing within 10 minutes to 24 hours under pressures within the range of normal pressure to 200 bar. In order to enrich rutin, isorhamnetin and/or precursors forming isorhamnetin further concentration steps such as liquid-liquid distribution using for example 1-butanol/water or ethylacetate/water, adsorption-desorption on ion exchangers, LH20, HP20 and other resins or chromatographic separations over RP18, silica gel and the like can be performed.

Rutin, isorhamnetin, isorhamnetin derivatives or plant parts and/or extracts produced therefrom which contain rutin or isorhamnetin in free or bonded form or from which isorhamnetin is formed by metabolization can be administered in form of powders, granules, tablets, dragees (coated tablets) or also as solutions, preferably orally.

For this purpose, rutin, isorhamnetin, isorhamnetin derivates or plant parts and/or extracts produced therefrom which contain rutin or isorhamnetin in free or bonded form or from which isorhamnetin is formed by metabolization, are mixed with suitable pharmaceutically acceptable adjuvants such as lactose, cellulose, silicon dioxide, croscarmellose and magnesium stearate and pressed into tablets which are optionally provided with a suitable coating which is made up of, for example, hydroxymethylpropylcellulose, polyethyleneglycol, colorants (e.g. titanium dioxide, iron oxide) and talcum.

The substances indicated above can also be filled into capsules, optionally under addition of adjuvants such as stabilizers, fillers and the like. The dosing is carried out such that 5 to 500 mg, preferably 20 to 200 mg rutin and/or isorhamnetin or the amount of an isorhamnetin derivative or plant extract which releases or contains the above amount of isorhamnetin are administered per day.

In case of those extracts which release isorhamnetin metabolically, the dosing is selected such that an isorhamnetin blood plasma level can be detected which corresponds to the isorhamnetin blood plasma level after administration of 5 to 500 mg, preferably 20 to 200 mg isorhamnetin.

The filling of the substances according to the present invention is preferably carried out together with oils and a particularly preferred embodiment comprises the filling of the extracts into capsules together with oils containing unsaturated fatty acids, preferably ω3 fatty acids, such as borage oil, evening primrose seed oil, fish oil, currant seed oil, linseed oil or perilla seed oil, thereby achieving an improvement in bioavailability of the active ingredients.

The efficacy of the above pure substances, plant parts and plant extracts in case of depressive diseases is evidenced by the experiments described in the following:

The antidepressive efficacy was examined by means of the so-called “forced swimming test” in rats. In performing this test rats are brought into an impasse situation (glass cylinder filled with water) over a specified period of 5 minutes. In this test the rats react with a rigor referred to as an immobilization period which is interpreted as a correlative to a depression. If the rats are treated with medicaments having an antidepressive efficacy before carrying out the test, the immobilization period is decreased. Since other psychotropic drugs such as anxiolytics or neuroleptics are not effective in this test, this test system is well suitable for detecting antidepressive effects (Porsolt et al. 1978; Porsolt, 1991). In this test all hitherto known antidepressives have to be administered over a period of several days in order to be effective (similar to the administration to patients). The test animals were treated either with the test substance or for control purposes with the solvent only or with the tricyclic antidepressive imipramine for a comparison of the efficacy. Imipramine was selected as a standard comparative substance because it is one of the most effective antidepressives both in psychiatric practice and in an animal model.

The following tables exemplarily show the efficacy of rutin, isorhamnetin and plant extracts containing isorhamnetin derivatives or rutin in sufficient amounts. In each table the inhibition of the immobility is expressed in terms of % inhibition against the control group for comparison purposes.

TABLE 1

| | Dosis | Inhibition of immobility |
|---|---|---|
| Substance | [mg/kg] | [% of the control] |
| Rutin | 3 | 8 |
| Rutin | 6 | 41 |
| Rutin | 12 | 42 |
| Rutin | 24 | 49 |
| Rutin | 48 | 74 |
| Imipramine | 30 | 46 |

TABLE 2

| | Dosis | Inhibition of immobility |
|---|---|---|
| Substance | [mg/kg] | [% of the control] |
| Isorhamnetin | 3 | 17 |
| Isorhamnetin | 10 | 56 |
| Imipramine | 3 | 3 |
| Imipramine | 10 | 24 |

TABLE 3

| | | | Isorhamnetin |
|---|---|---|---|
| | | Inhibition | [mg/kg] |
| | Dosis | [% of the | (determined after |
| Substance | [mg/kg] | control] | hydrolysis) |
| Extract from S. grandiflorus | 3 | 13 | 0.11 |
| (Example 1) | | | |
| Extract from S. grandiflorus | 10 | 30 | 0.36 |
| (Example 1) | | | |
| Extract from S. grandiflorus | 30 | 38 | 1.08 |
| (Example 1) | | | |
| Extract from S. grandiflorus | 100 | 48 | 3.60 |
| (Example 1) | | | |
| Imipramine | 20 | 43 | |

TABLE 4

| | Dosis | Inhibition | Rutin |
|---|---|---|---|
| Substance | [mg/kg] | [% of the control] | [mg/kg] |
| Extract from Ruta graveolens | 300 | 49 | 7.5 |
| (Example 2) | | | |
| Imipramine | 20 | 43 | |

TABLE 5

| | | | Isorhamnetin |
|---|---|---|---|
| | | | [mg/kg] |
| | | Inhibition | (determined |
| | Dosis | [% of the | after |
| Substance | [mg/kg] | control] | hydrolysis) |
| Extract from Opuntia (Example 3) | 10 | 16 | 0.44 |
| Extract from Opuntia (Example 3) | 30 | 34 | 1.32 |
| Extract from Opuntia (Example 3) | 100 | 52 | 4.40 |
| Extract from Opuntia (Example 3) | 300 | 83 | 13.20 |
| Imipramine | 30 | 64 | |

### EXAMPLES

### Example 1

### Extract from Selenicereus grandiflorus

200 g finely ground drug are stirred twice with 1400 g 60% by weight EtOH at 60° C. for 1 h, respectively. Subsequently, the suspension is filtered with suction over a frit P4, the combined filtrates are set free from EtOH in vacuum at 40° C., the remaining aqueous residue is freezed and lyophilised. The solid obtained is dried in vacuum at 40° C. over P₂O₅ and KOH: 30.2 g (15.1%) extract containing 0.1% isorhamnetin (determined without hydrolysis) and 3.6% isorhamnetin (determined after hydrolysis), respectively.

### Example 2

### Special Extract from Ruta graveolens

200 g finely ground dried leaves containing little blossoms are sequentially moved twice, using seven times their weight made up of n-heptane and subsequently twice using seven times their weight made up of ethylacetate for 1 h at 45° C. on a rotary evaporator, respectively. The suspensions obtained are filtered with suction over a frit P4 and the filtrates are laid aside. Then the drug residue is extracted twice as above, using 1400 g MeOH, respectively, and the combined filtrates are taken to dryness in vacuum at 40° C.: 33.71 g (16.9%) special extract containing 2.5% rutin.

### Example 3

### Extracts from Blossoms of Opuntia ficus-indica

2000 g finely ground drugs were extracted twice, using 14 kg 60% by weight EtOH for 1 h at 60° C., respectively. Subsequently, the suspension is filtered with suction over a nutsch filter, the combined filtrates are set free from aqueous ethanol at elevated temperature in vacuum and the remaining oil is dried in vacuum at 50° C.: 285 g (14.2%) extract containing 4.4% isorhamnetin (deter mined after hydrolysis).

### Example 4

### Tablets

An extract from Selenicereus grandiflorus is mixed with adjuvants and pressed tablets (core of the tablet=items 1-6). The tablets are provided with a hydroxypropylmethylcellulose coating (items 7-10).

| | Ingredient | mg/tablet |
|---|---|---|
| 1 | Extract from Selenicereus grandiflorus | 100.0 |
| 2 | Microcrystalline cellulose | 117.0 |
| 3 | Lactose monohydrate | 58.0 |
| 4 | Croscarmellose | 15.0 |
| 5 | Highly dispersed silicon dioxide | 3.0 |
| 6 | Magnesium stearate | 6.0 |
| 7 | Hydroxypropylmethylcellulose | 15.0 |
| 8 | Polyethyleneglycol | 3.0 |
| 9 | Talcum | 1.0 |
| 10 | Titanium dioxide | 2.0 |

### Example 5

### Capsules

An extract from Selenicereus grandiflorus is mixed with perilla seed oil and the flowable suspension obtained is filled into capsules according to a per se known method.

| | Ingredient | mg/filling of the capsules |
|---|---|---|
| 1 | Extract from Selenicereus grandiflorus | 100.0 |
| 2 | Perilla seed oil | 450.0 |

1-13. (canceled)

14. A method of treating a subject suffering from or susceptible to depression, depressive disease, other affective disorder and abjectness, feeling of reluctance, melancholia, unmotivation, labile or saddened mood, or to improve the mood of the subject, the method comprising:
administering to the subject isorhamnetin, isorhamnetin derivatives or plant parts and/or extracts produced therefrom which contain isorhamnetin in free or bonded form or from which isorhamnetin is formed by metabolization.

15. The method of claim 14 wherein isorhamnetin is formed by hydrolytic cleavage of an isorhamnetin derivative.

16. The method of claim 14 wherein the isorhamnetin derivative is a glycoside of isorhamnetin.

17. The method of claim 14 wherein the plant parts and/or the extracts are selected from plants of and/or an extract from Allium cepa, Anethum graveolens, Brassica oleracea, Cassia senna, Opuntia species, Peumus boldus, Primula veris, Sambucus nigra or Selenicereus grandiflorus.

18. The method of claim 14 wherein the plant parts and/or the extracts are produced from blossoms of Selenicereus grandiflorus.

19. The method of claim 14 wherein wherein the plant parts and/or the extracts are produced from blossoms of Opuntia ficus-indica.

20. The method of claim 14 wherein a pharmaceutical composition is administered that comprises oil which contains unsaturated fatty acids.

21. The method of claim 20 wherein the oil is selected from the group comprising borage oil, evening primrose seed oil, currant seed oil, fish oil, linseed oil and perilla seed oil.

22. A pharmaceutical composition comprising isorhamnetin, isorhamnetin derivatives or plant parts and/or extracts produced therefrom, which contain isorhamnetin in free or bonded form or from which isorhamnetin is formed by metabolization.

23. The pharmaceutical composition of claim 22 further comprising an oil containing unsaturated fatty acids.

24. The pharmaceutical composition of claim 23 wherein the oil is selected from the group comprising borage oil, evening primrose seed oil, currant seed oil, fish oil, linseed oil and perilla seed oil.

25. A dietetic food product comprising isorhamnetin, isorhamnetin derivatives or plant parts and/or extracts produced therefrom, which contain isorhamnetin in free or bonded form or from which isorhamnetin is formed by metabolization.

26. The dietetic food product of claim 25 further comprising an oil containing unsaturated fatty acids.

27. The dietetic food product of claim 26 wherein the oil is selected from the group comprising borage oil, evening primrose seed oil, currant seed oil, fish oil, linseed oil and perilla seed oil.
